(19) United States
(12) Patent Application Publication
(10) Pub. No.: US 2021/0368880 A1
(43) Pub. Date: Dec. 2, 2021

(54) DETACHABLE CAPTURE DEVICE FOR BIOMATERIALS

(71) Applicant: KOREA GENE INFORMATION INSTITUTE CO., LTD, Seoul (KR)

(72) Inventors: Young Deug Kim, Seoul (KR); Byeong Joo Park, Seoul (KR); Jin Wook Jung, Seoul (KR); Seung Bum Hong, Seoul (KR); Sang min Lee, Seoul (KR)

(21) Appl. No.: 17/099,437

(22) Filed: Nov. 16, 2020

Related U.S. Application Data
(63) Continuation of application No. PCT/KR2020/008481, filed on Jun. 29, 2020.

(30) Foreign Application Priority Data
Jun. 2, 2020 (KR) 10-2020-0066650
Jun. 15, 2020 (KR) 10-2020-0072426

(51) Int. Cl. A41D 13/11

(52) U.S. Cl. CPC A41D 13/1176; A41D 13/1107; A41D 2300/32; A41D 2600/00; A41D 2500/40; A41D 2500/30; A41D 2300/20

(57) ABSTRACT

A detachable capture device for biomaterials may include a capture unit and an attachment unit. The capture unit may have a certain area to be attached biomaterials that causes respiratory diseases thereon, and the attachment unit may be detachably attached to an inner surface of the mask or an outer surface of the mask to fix the capture unit to the mask.

### CROSS-REFERENCE TO RELATED APPLICATIONS

This application is a continuation of PCT/KR2020/008481, filed Jun. 29, 2020, herein incorporated by reference in its entirety, which claims priority under 35 USC § 119 to Korean Patent Application No. 10-2020-0066650, filed on Jun. 2, 2020 and Korea Patent Application No. 10-2020-0072426, filed on Jun. 15, 2020 before the Korean Intellectual Property Office (KIPO), the contents of which are herein incorporated by reference in their entirety.

### BACKGROUND

### 1. Field

The present application relates to a detachable capture device for biomaterials. More particularly, the present application relates to a capture device for biomaterials of easily collecting biomaterials regarding respiratory diseases by being detachable on a mask.

### 2. Description of the Related Art

Due to recent global pandemic of COVID-19, various methods have been attempted to block the spread of the causative agent (hereinafter referred to as ‘biomaterial’) that causes respiratory diseases. The biomaterials include bacteria and virus, e.g., Influenza Virus, Corona Virus, Respiratory Syncytial Virus, or the like, and are mainly spreaded through small droplets such as saliva generated when coughing or sneezing. Thus, a mask is widely used as a tool that can easily and effectively block droplet to cause spread of infection.

On the other hand, methods for checking whether or not biomaterials are actually on the mask surface include a smear method in which biomaterials are collected by scraping the mask surface with cotton swab, and a method of cutting out a part of the mask surface and inspecting it. However, these methods are inconvenient to prepare a medical cotton swab or medical scissors. Accordingly, there is a need for a method capable of easily collecting biomaterials on the mask surface without using a medical tool.

### SUMMARY

Example embodiments provide a detachable capture device for biomaterials that can be easily attached to and detached from a mask, so that biomaterials adsorbed to the inner or outer surface of a mask on the market can be easily collected without using a medical tool.

According to example embodiments, a detachable capture device for biomaterials includes a capture unit having a certain area to be attached biomaterials that causes respiratory diseases thereon, and an attachment unit detachably attached to an inner surface of the mask or an outer surface of the mask to fix the capture unit to the mask.

In example embodiments, the capture unit includes at least one of paper, nonwoven fabric, and cotton cloth. And the capture unit includes two or more types of fibers having different fineness.

In example embodiments, the attachment unit includes an attachment body, a support, and an adhesive. The attachment body contacts to the inner surface or the outer surface of the mask, and has an opening therethrough. The support connects the attachment body on both sides of the opening across the opening. The adhesive is partially or entirely applied to the rear surface of the attachment body to detachably fix the attachment body to the inner or outer surface of the mask.

In example embodiments, an upper portion and a lower portion of the capture unit are in contact with the rear surface of the attachment body, respectively. And at least a portion between the upper and lower portions of the capture unit is in contact with the front surface of the support.

In example embodiments, the attachment unit is an adhesive that is partially or entirely applied to the rear surface of the attachment body.

In example embodiments, the capture unit and the attachment unit are coupled by a hook-and-loop fastener method.

According to another example embodiments, a detachable capture device for biomaterials includes a capture unit having a certain area to be attached biomaterials that causes respiratory diseases thereon, an attachment unit detachably attached to an inner surface of the mask or an outer surface of the mask to support the capture unit to the mask, and a cover covering a front surface of the capture unit and the attachment unit.

In example embodiments, the capture unit includes at least one of paper, nonwoven fabric, and cotton cloth. And the capture unit includes two or more types of fibers having different fineness.

In example embodiments, wherein the attachment unit surrounds the circumference of the capture unit. Here, the thickness of the attachment unit is thicker than the thickness of the capture unit, so that the capture unit is spaced apart from the mask and the cover.

In example embodiments, the cover includes a cutout portion in which a plurality of through holes spaced apart from each other are arranged linearly.

### BRIEF DESCRIPTION OF THE DRAWINGS

Example embodiments will be more clearly understood from the following detailed description taken in conjunction with the accompanying drawings. FIGS. 1 to 5 represent non-limiting, example embodiments as described herein.

FIG. 1 is a diagram illustrating a mask attaching a capture device for biomaterials thereon in accordance with example embodiments;

FIG. 2 is a diagram illustrating the capture device for biomaterials in FIG. 1;

FIG. 3 is a diagram illustrating a biomaterial collection kit including the capture device for biomaterials in FIG. 1;

FIG. 4 is a diagram illustrating the capture device for biomaterials in accordance with example embodiments; and

FIG. 5 is a diagram illustrating the capture device for biomaterials in accordance with example embodiments.

### DESCRIPTION OF EMBODIMENTS

With respect to the embodiments of the present invention disclosed hereinafter, specific structural or functional descriptions have been exemplified for the purpose of describing the embodiments of the present invention only, and the embodiments of the present invention may be implemented in various forms. It should not be construed as being limited to the embodiments set forth herein.

Since the present invention can apply various changes and have various forms, specific embodiments will be illustrated in the drawings and described in detail in the text. However, this is not intended to limit the present invention to a specific form of disclosure, it is to be understood as including all changes, equivalents, or substitutes included in the scope of the present invention.

It will be understood that, although the terms first, second, third, fourth etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive concept.

It will be understood that when an element is referred to as being “connected to” or “coupled to” another element, it can be directly on, connected or coupled to the other element or intervening elements may be present. In contrast, when an element is referred to as being “directly connected to” or “directly coupled to” another element, there are no intervening elements present. Other expressions describing the relationship between components, such as “between” and “just between” or “adjacent to” and “directly adjacent to” should be interpreted as well.

The terms used in the present invention are only used to describe specific embodiments, and are not intended to limit the present invention. Singular expressions include plural expressions unless the context clearly indicates otherwise. In the present invention, terms such as “comprise” or “have” are intended to designate the presence of a set feature, number, step, action, component, part, or combination thereof, but it is to be understood that one or more other features or numbers the possibility of addition or presence of, steps, actions, components, parts, or combinations thereof is not preliminarily excluded.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, preferred embodiments of the present invention will be described in more detail with reference to the accompanying drawings. The same reference numerals are used for the same elements in the drawings, and duplicate descriptions for the same elements are omitted.

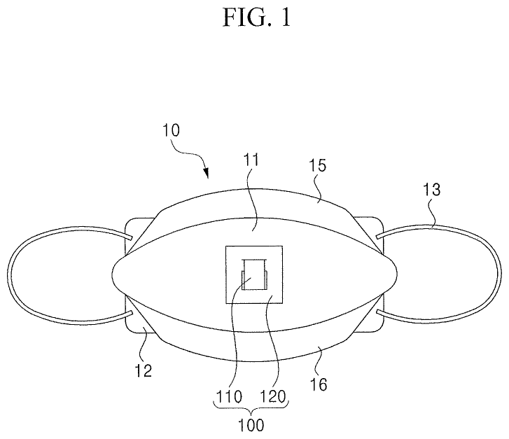

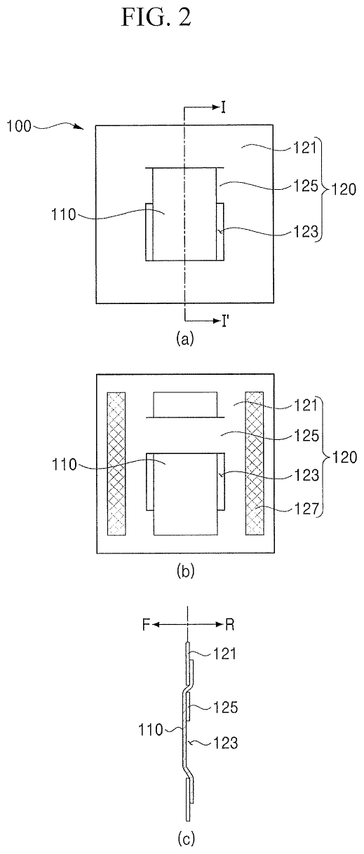

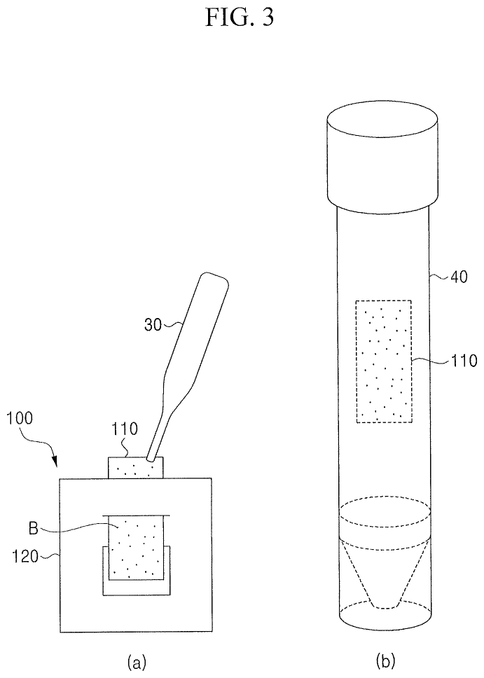

FIG. 1 is a diagram illustrating a mask attaching a capture device for biomaterials thereon in accordance with example embodiments. FIG. 2 is a diagram illustrating the capture device for biomaterials in FIG. 1. FIG. 3 is a diagram illustrating a biomaterial collection kit including the capture device for biomaterials in FIG. 1.

Here, the term ‘biomaterial’ refers to a substance that causes various respiratory diseases, such as proteins, lipids, and nucleic acids. For example, the biomaterial includes bacteria, influenza virus, corona virus, respiratory syncytial virus, and the like. For convenience of explanation, hereinafter, the causative agent that causes respiratory disease will be simply referred to as a ‘biomaterial’.

Referring to FIG. 1, a mask 10 may include an inner surface 11 covering the wearer's respiratory tract, an outer surface 12 attached to the back of the inner surface 11 and having a shape corresponding to the inner surface 11, a filter (not illustrated) inserted between the inner surface 11 and the outer surface 12 to remove biomaterials or fine dust, and a pair of circular shaped bands 13 provided on both sides of the inner surface 11 or the outer surface 12 to be hung on the wearer's ear. The wearer may wear the mask 10 by hanging the band 13 on the ear while the inner surface 11 of the mask 10 is in close contact with the face to cover the respiratory tract. In FIG. 1, the mask 10 is a three-stage folding-type mask in which a nose pad 15 on the upper part of the mask 10 and a bib 16 on the lower part of the mask 10 are folded, but the present invention is not limited thereto. That is, the present invention can be applied equally or similarly to various types of masks, e.g., two-stage folding-type mask that is folded left and right sides, a dental mask that is folded in multiple stages, or the like. In addition, the present invention can also be applied equally or similarly to an object which contamination by biomaterials is concerned.

Meanwhile, in FIG. 1, the capture device for biomaterials 100 is attached to the inner surface 11 of the mask 10, but it is not be limited thereto. For example, the capture device for biomaterials 100 may be selectively attached to the outer surface 12 of the mask 10 instead of the inner surface 11 of the mask 10. When the capture device for biomaterials 100 is attached to the inner surface 11 of the mask 10, it can be checked whether the biomaterials derived from the wearer's saliva is stained on the mask 10. Alternatively, when the capture device for biomaterials 100 is attached to the outer surface 12 of the mask 10, it can be checked whether the biomaterials derived from the outside is stained on the mask 10. Hereinafter, for the convenience of description, only an embodiment in which the capture device for biomaterials 100 is attached to the inner surface 11 of the mask 10 will be described.

Referring to FIGS. 1 to 3, the capture device of biomaterials 100 may include a capture unit 110 capturing the biomaterials, and an attachment unit 120 attached to the surface of the mask 10 to fix the capture unit 110 to the surface of the mask 10.

The capture unit 110 may be made of a material capable of easily capture biomaterials, e.g., paper, nonwoven fabric, cotton cloth, or the like to have a certain area. The capture unit 110 may be selectively fixed to the inner surface 11 of the mask 10 or the outer surface 12 of the mask 10 by the attachment unit 120. Accordingly, when the droplet is sprayed toward the inner surface 11 of the mask 10 or the outer surface 12 of the mask 10, biomaterials included in the sprayed droplet may be attached to the surface of the capture unit 110.

The shape or size of the capture unit 110 may be appropriately changed, as necessary. For example, as shown in FIG. 2, when the capture unit 110 has a rectangular shape of a narrow width, the capture unit 110 can be easily accommodated in the virus transport medium 40 (see FIG. 3). On the other hand, the larger the area of the capture unit 110 is, the wider the area of the mask 10 may be covered.

In some example embodiments, the capture unit 110 may include two or more types of fibers having different fineness, respectively. The fineness means the thickness of the fiber. When a nonwoven fabric or cotton fabric is manufactured by mixing a plurality of fibers having different fineness, the density of the capture unit 110 may be increased, which leads to an increase in the capture efficiency of biomaterials. Accordingly, the capture efficiency of the biomaterial may be improved by making the capture unit 110 by mixing two or more types of fibers having different fineness.

In some example embodiments, to maximize the capture efficiency of saliva or virus, polysaccharides with adhesive properties may be applied to the capture unit 110. Here, the polysaccharides are water-soluble polysaccharides formed by regularly repeating 1 to 6 monosaccharide subunits in a linear or linear form having some branches, and the polysaccharides may have a molecular weight of 10 to 200 kDa. Preferably, the polysaccharides may include any one of xanthan gum, dextran, welan gum, gellan gum, diutan gum, pullulan, hyaluronan, alginic acid, carrageenan, agar gum, guar gum, karaya gum, locust bean gum, and tamarind gum, or may include a mixture thereof. More preferably, the polysaccharides may be a mixture of carrageenan, alginic acid, and xanthan gum in a ratio of 1:0.1˜100:1:0.01˜100.

In some example embodiments, the capture unit 110 may be coated with 0.01% (w/v) to 0.1% (w/v) of an aqueous solution containing the polysaccharide. That is, by performing a pretreatment process of applying the polysaccharide aqueous solution to the capture unit 110 and drying it, the capture efficiency of the capture unit 110 may be improved.

The attachment unit 120 may be attached to the surface of the mask 10 to fix the capture unit 110 to the surface of the mask 10.

As shown in FIG. 2, the attachment unit 120 may include an attachment body 121 in contact with the surface of the mask 10, an opening 123 penetrating the attachment body 121 to expose the capture unit 110, a support 125 crossing the opening 123 to support the rear surface of the capture unit 110, and an adhesive 127 firmly fixing the attachment body 127 to the surface of the mask 10.

The attachment body 121 may be made of a various material, e.g., paper, nonwoven fabric, cotton cloth, plastic film, or the like. Preferably, in order to remove feeling of irritation when wearing the mask 10, the attachment body 121 may be made of the same material as the inner surface 11 of the mask 10 or the outer surface 12 of the mask 10.

The opening 123 may be formed by removing a part of the attachment body 121, and may provide a space in which the capture unit 110 is exposed to the outside. Thus, the width and length of the opening 123 may be appropriately changed according to the size and shape of the capture unit 110.

By the way, in order to fix the capture unit 110 to the attachment unit 120 without any other structure supporting the capture unit 110 besides the opening 123, one side of the capture unit 110 have to contact with the rear surface of the attachment body 121, and the other side of the capture unit 110 have to contact with the front surface of the attachment body 121. Here, ‘the front surface’ refers to a surface located in the direction F toward the wearer's face, and ‘the rear surface’ refers to a surface located in the direction R toward the surface of the mask 10.

However, in such a coupling form, since a sufficient coupling force between the capture unit 110 and the attachment unit 120 cannot be expected, an adhesive or the like must be additionally used. If the capture unit 110 is fixed using an adhesive, it may be difficult to separate the capture unit 110 from the attachment unit 120 after collecting the biomaterials. Further, since the rear surface of the capture unit 110 is contaminated with the adhesive, accurate inspection may be difficult. To solve this problem, in the present invention, the support 125 crossing the opening 123 to connect the capture unit 110 on both sides of the opening 123 is additionally formed. In this case, the capture unit 110 sequentially contacts to a rear surface of the attachment body 121, an upper surface of the support 125, and a rear surface of the attachment body 121 along the longitudinal direction of the capture unit 110. That is, both sides of the capture unit 110 contact to the rear surface of the attachment body 121, and thus sufficient bonding force can be secured between the capture unit 110 and the attachment unit 120 even if an adhesive is not used.

The capture unit 110 may be detachably coupled to the attachment unit 120, which is shown in FIG. 3. As shown in FIG. 3, the capture unit 110 in which biomaterials are captured therein may be easily separated from the attachment unit 120 using tweezers 30 or the like. The separated capture unit 110 may be accommodated in the virus transport medium 40 and transported to the analysis site. That is, a biomaterial collection kit for collecting the biomaterials B attached on the mask 10 and transporting it to the laboratory for analysis, may be composed of the capture device for biomaterials 100, tweezers 30, and the virus transport medium 40.

The adhesive may be partially or entirely applied to the rear surface of the attachment body 121. The adhesive may firmly fix the attachment body 121 to the inner surface 11 of the mask 10 or the outer surface 12 of the mask 10.

By the way, in FIGS. 1 to 3, the attachment unit 120 is firmly attached to the surface of the mask 10 and the capture unit 110 is detachably coupled to the attachment unit 120, but the present invention is not limited thereto. In some example embodiments, the capture device for biomaterials 100 may be composed of only the capture unit 110 without the attachment unit 120.

For example, by partially or entirely applying an adhesive to the rear surface of the capture unit, the capture unit 110 may be directly attached to the inner surface 11 of the mask 10 or the outer surface 12 of the mask 10. In this case, the capture device for biomaterials can be downsized.

As described above, the capture device for biomaterials 100 according to the present invention may be detachably attached to the inner surface 11 of the mask 10 or the outer surface 12 of the mask 10. When the capture device for biomaterials 100 is attached to the inner surface 11 of the mask 10, the biomaterials B included in the mask wearer's saliva or the like can be easily collected. Alternatively, when the capture device for biomaterials 100 is attached to the outer surface 12 of the mask 10, the biomaterials B introduced into the mask 10 from the outside can be easily collected. In particular, the present invention can be easily applied to various types of masks sold on the market and can be manufactured at a low price. Accordingly, it is possible to more effectively prevent the spread of respiratory diseases by easy sample collection.

Additionally, the present invention can easily check the degree of exposure to the biomaterials B in the usual living environment wearing the mask 10 without visiting a respiratory disease testing facility such as a hospital or a screening clinic. Particularly, in case of COVID-19, one of the serious problems is asymptomatic infection, and there is a problem that it is difficult to expect an asymptomatic infected person to voluntarily visit specialized testing facilities. However, if the capture device for biomaterials 100 according to the present invention is used, anyone can inexpensively and easily collect the biomaterial B stained on the mask 10 without visiting a specialized inspection facility. Accordingly, samples of asymptomatic infected individuals can be easily collected to prevent the spread of respiratory diseases.

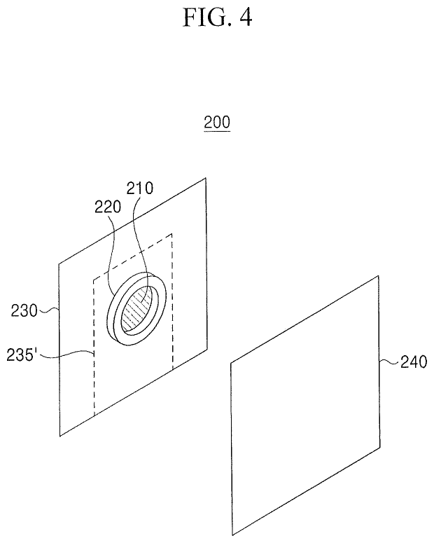

Hereinafter, another embodiment of the present invention having a patch type will be described with reference to FIG. 4.

FIG. 4 is a diagram illustrating the capture device for biomaterials in accordance with example embodiments.

As shown in FIG. 4, the capture device for biomaterials 200 may include a capture unit 210 capturing the biomaterials B, an attachment unit 220 detachably attached to the inner surface 11 or the outer surface 12 of the mask 10 to fix the capture unit 210 to the surface of the mask 10, a cover 230 covering a front surface of the attachment unit 220, and a protecting film 240 covering a rear surface of the attachment unit 220 and the cover 230.

The capture unit 210 may be made of a material capable of easily capture biomaterials, e.g., paper, nonwoven fabric, cotton cloth, or the like, and may be selectively fixed to the inner surface 11 of the mask 10 or the outer surface 12 of the mask 10 by the attachment unit 220. The role, material, and shape of the capture unit 210 are the same as or similar to those of the capture unit 110 described with reference to FIGS. 1 to 3, and thus a redundant description thereof will be omitted.

The attachment unit 220 may surround the circumference of the capture unit 210. The attachment unit 220 may attached to the inner surface 11 or the outer surface 12 of the mask 10 to fix the capture unit 210 to the surface of the mask 10. Here, the thickness of the attachment unit 220 may be thicker than the thickness of the capture unit 210. That is, the front surface of the attachment unit 220 may contact the rear surface of the cover 230, and the rear surface of the attachment unit 220 may contact to the inner surface 11 or the outer surface 12 of the mask 10. And due to the thickness of the attachment unit 220, the capture unit 210 may not contact both of the cover 230 and the surface of the mask 10. Accordingly, the attachment unit 220 may prevent contamination of the capture unit 210 by preventing the capture unit 210 from directly contacting the cover 230 and the surface of the mask 10.

Meanwhile, in FIG. 4, an embodiment in which both the capture unit 210 and the attachment unit 220 have circular shape is shown, but the present invention is not limited thereto. That is, the shapes of the capture unit 210 and the attachment unit 220 may be appropriately changed as necessary, such as an oval, a square, a polygon, or the like.

The cover 230 cover a front surface of the capture unit 210 and the attachment unit 220. Here, ‘the front surface’ refers to a surface located in the direction toward the wearer's face. That is, the cover 230 may cover the front surface of the capture unit 210 to prevent the capture unit 210 from directly contact the wearer of the mask 10. At this time, the attachment unit 220 and the cover 230 may be firmly bonded with an adhesive or the like.

In some example embodiments, a cutout portion 235 for easily separating the attachment unit 220 from the cover 230 may be formed in the cover 230. For example, the cutout portion 235 may be formed in which small through holes penetrating the cover 230 are linearly and densely arranged to include at least the outer boundary of the attachment unit 220. In this case, after the biomaterial B is sufficiently collected, a part of the cover 230 along the cutout portion 235 may be easily removed by applying force to the cutout portion 235 with tweezers 30 or the like. Therefore, even if the entire cover 230 is not removed, the capture unit 210 and the attachment unit 220 can be easily separated from the cover 230.

Meanwhile, an adhesive may be partially or entirely applied to the rear surface of the cover 230 or the rear surface of the attachment unit 220. Due to the adhesive, the capture device for biomaterials 200 may be detachably attached to the surface of the mask 10. The protecting film 240 may be bonded to the rear surface of the cover 230 and the attachment unit 220 in a state before the start of use to preserve the adhesive performance of the adhesive, and to prevent contamination of the capture unit part 210.

As illustrated above, the capture device for biomaterials 200 according to the present invention may be detachably attached to the inner surface 11 of the mask 10 or the outer surface 12 of the mask 10. Accordingly, the biomaterials B on the inner surface 11 or the outer surface 12 of the mask 10 can be easily collected. In particular, the present invention can be easily applied to various types of masks sold on the market and can be manufactured at a low price. Accordingly, it is possible to more effectively prevent the spread of respiratory diseases by easy sample collection.

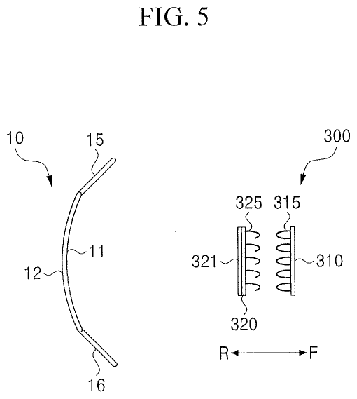

Hereinafter, another embodiment of the present invention will be described with reference to FIG. 5.

The capture device for biomaterials 300 of FIG. 5 is substantially the same as or similar to the capture device for biomaterials 100 described with reference to FIGS. 1 to 3 except for a coupling method between the capture unit 310 and the attachment unit 320. Therefore, only the above differences will be described, and duplicate descriptions of the same components will be omitted.

Referring to FIG. 5, the capture device for biomaterials 300 may include a capture unit 310 capturing the biomaterials B, and an attachment unit 320 detachably attached to the inner surface 11 or the outer surface 12 of the mask 10 to support the capture unit 310.

The capture unit 310 may be made of a material capable of easily capture biomaterials, e.g., paper, nonwoven fabric, cotton cloth, or the like. Polysaccharides with adhesive properties may be applied to the surface of the capture unit 310.

The attachment unit 320 may detachably attached to the inner surface 11 or the mask 10 or the outer surface 12 of the mask 10. For example, an adhesive may be partially or entirely applied to the rear surface of the attachment unit 320. Here, ‘the rear surface’ refers to a surface located in the direction R toward the surface of the mask 10, and ‘the front surface’ refers to a surface located in the direction F toward the capture unit 310.

In some example embodiments, the capture unit 310 and the attachment unit 320 may be detachably coupled with a hook-and-loop fastener method widely known under the trade name of Velcro.

For example, a loop 315 may be provided on the rear surface (a surface facing the R direction) of the capture unit 310, and a hook 325 may be provided on the front surface (a surface facing the F direction) of the attachment unit 320. Since the hook 325 is coupled to the loop 315, the capture unit 310 can be easily attached to the attachment unit 320 without using a chemical adhesive. In addition, it is possible to easily separate the capture unit 310 from the attachment unit 320 with little force.

Alternatively, a hook may be provided on the rear surface of the capture unit 310, and a loop may be provided on the front surface of the attachment unit 320.

As illustrated above, the capture device for biomaterials 300 according to the present invention may be detachably attached to the inner surface 11 of the mask 10 or the outer surface 12 of the mask 10. Accordingly, the biomaterials B on the inner surface 11 or the outer surface 12 of the mask 10 can be easily collected. In particular, since the capture unit 310 for collecting the biomaterials is coupled by a hook-and-loop fastener method, it can be easily attached and detached without using a chemical adhesive.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept.

Additionally, although it is described that the capture device for biomaterials of the present invention is attached to the mask, but it is not be limited thereto. The capture device for biomaterials also used for monitoring by attaching it to a place where it is necessary to collect biomaterials related to respiratory diseases other than a mask, e.g., karaoke microphones, personal computer, interior of sealed vehicles, air conditioner vent of the school or group's catering room, a vent of a building or an office, a vent of public facilities, or the like.

What is claimed is:

1. A detachable capture device for biomaterials, comprising:
a capture unit having a certain area to be attached biomaterials that causes respiratory diseases thereon; and
an attachment unit detachably attached to an inner surface of the mask or an outer surface of the mask to fix the capture unit to the mask.

2. The detachable capture device for biomaterials of claim 1, wherein the capture unit includes at least one of paper, nonwoven fabric, and cotton cloth,
and wherein the capture unit includes two or more types of fibers having different fineness.

3. The detachable capture device for biomaterials of claim 1, wherein the attachment unit includes,
an attachment body in contact with the inner surface or the outer surface of the mask, and having an opening therethrough;
a support for connecting the attachment body on both sides of the opening across the opening; and
an adhesive that is partially or entirely applied to the rear surface of the attachment body to detachably fix the attachment body to the inner or outer surface of the mask.

4. The detachable capture device for biomaterials of claim 3, wherein an upper portion and a lower portion of the capture unit are in contact with the rear surface of the attachment body, respectively, and at least a portion between the upper and lower portions of the capture unit is in contact with the front surface of the support.

5. The detachable capture device for biomaterials of claim 1, wherein the attachment unit is an adhesive that is partially or entirely applied to the rear surface of the attachment body.

6. The detachable capture device for biomaterials of claim 1, wherein the capture unit and the attachment unit are coupled by a hook-and-loop fastener method.

7. A detachable capture device for biomaterials, comprising:
a capture unit having a certain area to be attached biomaterials that causes respiratory diseases thereon;
an attachment unit detachably attached to an inner surface of the mask or an outer surface of the mask to support the capture unit to the mask; and
a cover covering a front surface of the capture unit and the attachment unit.

8. The detachable capture device for biomaterials of claim 7, wherein the capture unit includes at least one of paper, nonwoven fabric, and cotton cloth,
and wherein the capture unit includes two or more types of fibers having different fineness.

9. The detachable capture device for biomaterials of claim 7, wherein the attachment unit surrounds the circumference of the capture unit,
and wherein the thickness of the attachment unit is thicker than the thickness of the capture unit, so that the capture unit is spaced apart from the mask and the cover.

10. The detachable capture device for biomaterials of claim 9, wherein the cover includes a cutout portion in which a plurality of through holes spaced apart from each other are arranged linearly.
